(19) United States
(12) Patent Application Publication
(10) Pub. No.: US 2008/0269242 A1
(43) Pub. Date: Oct. 30, 2008

(54) Crystalline Salts of 7-[4-(4-Naphthalen-1-Yl-Piperazin-1-Yl)-Butoxy]-3,4-Dihydro-1H-[1,8]Naphthyridine-2-One

(76) Inventors: Vladimir Genukh Beylin, Ann Arbor, MI (US); Nahid Yahyai, Superior Township, MI (US)

Correspondence Address: PFIZER INC;Steve T. Zelson, 150 EAST 42ND STREET, 5TH FLOOR - STOP 49, NEW YORK, NY, 10017-5612, US

(21) Appl. No.: 12/090,262

(22) PCT Filed: Oct. 18, 2006
(86) PCT No.: PCT/IB06/02971

§ 371 Date: Apr. 15, 2008

Related U.S. Application Data
(60) Provisional application No. 60/731,919, filed on Oct. 31, 2005.

(51) Int. Cl. A61K 31/496; C07D 471/04; A61P 25/18

(52) U.S. Cl. 514/253.04; 544/362

(57) ABSTRACT

The present application relates to crystalline salts of 7-[4-(4-naphthalen-1-yl-piperazin-1-yl)-butoxy]-3,4-di-hydro-1H-[1,8]naphthyridin-2-one, process for preparation of the crystalline salts, pharmaceutical compositions containing the crystalline salts, and use of the crystalline salts for treating certain disorders or conditions.

### FIELD OF THE INVENTION

The present invention relates to [1,8]naphthyridin-2-one compounds, and more specifically, to crystalline salts of 7-[4-(4-naphthalen-1-yl-piperazin-1-yl)-butoxy]-3,4-dihydro-1H-[1,8]naphthyridin-2-one (TUPAC name), which is also known by the CAS name 3,4-dihydro-1,8-naphthyridin-2(1H)-one,7-[4-[4-(1-naphthalenyl)-1-piperazinyl]-butoxy].

### BACKGROUND OF THE INVENTION

WO2005/019215 discloses the compound 7-[4(4-naphthalen-1-yl-piperazin-1-yl)-butoxy]-3,4-dihydro-1H-[1,8]naphthyridin-2-one, a process for preparing the compound, pharmaceutical compositions containing the compound, and the use of the compound for treating for certain disorders or conditions. The full disclosure of W02005/019215 is incorporated herein by reference. The compound 7-[4-(4-naphthalen-1-yl-piperazin-1-yl)-butoxy]-3,4dihydro-1H-[1,8]naphthyridin-2-one, which is represented by the structural formula

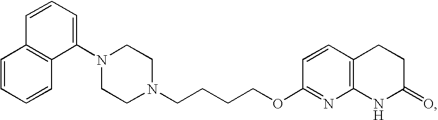

binds to the dopamine D₂ receptor and exhibits activity as a partial agonist of D₂ receptors. As such, 7-[4-(4-naphthalen-1-yl-piperazin-1-yl)-butoxy]-3,4-dihydro-1H-[1,8]naphthyridin-2-one, as well as salts thereof, is useful for the treatment of schizophrenia and other central nervous system disorders.

### BRIEF DESCRIPTION OF THE DRAWINGS

FIG. 1. Powder X-ray diffraction pattern of crystalline Form A 7-[4-(4-naphthalen-1-yl-piperazin-1-yl)-butoxy]-3,4-dihydro-1H-[1,8]naphthyridin-2-one mono-phosphate. The powder X-ray diffraction pattern was measured on a Bruker D8 Discover X-ray powder diffractometer with a GADDS CS operating in reflection mode using Cu Kα radiation (1.54).

FIG. 2. Powder X-ray diffraction pattern of crystalline form B 7-[4-(4-naphthalen-1-yl-piperazin-1-yl)-butoxy]-3,4-dihydro-1H-[1,8]naphthyridin-2-one mono-phosphate. The powder X-ray diffraction pattern was measured on a Bruker D8 Discover X-ray powder diffractometer with a GADDS CS operating in reflection mode using Cu Kα radiation (1.54).

### SUMMARY OF SPECIFIC EMBODIMENTS OF THE INVENTION

In one aspect of the invention, there is provided a crystalline 7-[4-(4-naphthalen-1-yl-piperazin-1-yl)-butoxy]-3,4-dihydro-1H-[1,8]naphthyridin-2-one mono-phosphate salt. In one embodiment, there is provided a crystalline 7-[4-(4-naphthalen-1-yl-piperazin-1-yl)-butoxy]-3,4-dihydro-1H-[1,8]naphthyridin-2-one mono-phosphate having a powder X-ray diffraction pattern shown in FIG. 1 or expressed in terms of 2-theta (2θ), d-spacing, and relative intensity of all peaks having a relative intensity greater than 10%, as provided in Table 1. In another embodiment, there is provided a crystalline 7-[4-(4naphthalen-1-yl-piperazin-1-yl)-butoxy]-3,4-dihydro-1H-[1,8]naphthyridin-2-one one mono-phosphate having a powder X-ray diffraction pattern shown in FIG. 2 or expressed in terms of 2-theta, d-spacing, and relative intensities all peaks having a relative intensity greater than 10%, as provided in Table 2.

In another aspect of the invention, there is provided a process of for the preparation of crystalline 7-[4-(4-naphthalen-1-yl-piperazin-1-yl)-butoxy]-3,4-dihydro-1H-[1,8]naphthyridin-2-one mono-phosphate.

In a further aspect of the invention, there is provided a method of treating a disorder or condition in a mammal wherein stimulation of D₂ receptor is beneficial, which method comprises administering to the mammal an effective amount of a crystalline 7-[4-(4-naphthalen-1-yl-piperazin-1-yl)-butoxy]-3,4-dihydro-1H-[1,8]naphthyridin-2-one mono-phosphate.

In yet another aspect of the invention, there is provided a pharmaceutical composition which comprises a therapeutically effective amount of a crystalline 7-[4-(4-naphthalen-1-yl-piperazin-1-yl)-butoxy]-3,4-dihydro-1H-[1,8]naphthyridin-2-one mono-phosphate salt and a pharmaceutically acceptable carrier.

### DETAILED DESCRIPTION OF SPECIFIC EMBODIMENTS OF THE INVENTION

In one aspect of the invention, there is provided a crystalline 7-[4(4-naphthalen-1-yl-piperazin-1-yl)-butoxy]-3,4-dihydro-1H-[1,8]naphthyridin-2-one mono-phosphate salt.

In one embodiment, there is provided a crystalline 7-[4(4-naphthalen-1-yl-piperazin-1-yl)-butoxy]-3,4-dihydro-1H-[1,8]naphthyridin-2-one monophosphate having a powder X-ray diffraction pattern shown in FIG. 1 or expressed in terms of 2-theta (20), d-spacing, and relative intensity of all peaks having a relative intensity greater than 10% as provided in Table 1, wherein the powder X-ray diffraction pattern was measured on a Bruker D8 Discover X-ray powder diffractometer with a GADDS CS operating in reflection mode using Cu Kα radiation (1.54). For the sake of identification this crystalline form of 7-[4-(4-naphthalen-1-yl-piperazin-1-yl)-butoxy]-3,4-dihydro-1H-[1,8]naphthyridin-2-one mono-phosphate is designated as “crystalline Form A or “Form A.

TABLE 1
2-Theta, d-Spacing, and Relative Intensity of All Peaks with Relative
Intensities Greater than 10% for Form A

| 2-Theta | d-Spacing | Relative Intensity (%) |
|---|---|---|
| 9.7 | 9.12225 | 23.8 |
| 10.2 | 8.64029 | 10.4 |
| 11.6 | 7.62229 | 8.5 |
| 12.0 | 7.36909 | 15.1 |
| 12.4 | 7.14407 | 31.3 |
| 12.9 | 6.88349 | 12.0 |
| 14.6 | 6.07379 | 63.1 |
| 15.3 | 5.80509 | 17.6 |
| 15.9 | 5.57742 | 57.1 |
| 16.3 | 5.42339 | 100.0 |
| 18.1 | 4.89432 | 27.3 |
| 18.7 | 4.74490 | 69.2 |
| 19.3 | 4.60243 | 73.0 |
| 19.9 | 46569 | 33.7 |
| 20.5 | 4.32202 | 79.7 |
| 22.1 | 4.02297 | 72.6 |
| 23.1 | 3.8481 | 58.9 |
| 23.7 | 3.74722 | 47.3 |
| 24.4 | 3.63859 | 35.8 |
| 25.5 | 3.48674 | 33.1 |
| 26.5 | 3.36356 | 24.5 |
| 27.5 | 3.24616 | 16.5 |
| 28.1 | 3.1705 | 14.6 |
| 29.4 | 3.04023 | 33.1 |
| 31.1 | 2.87324 | 21.7 |
| 31.5 | 2.83775 | 17.4 |
| 32.9 | 2.71797 | 25.1 |
| 34.4 | 2.60278 | 18.9 |
| 35.8 | 2.50349 | 15.9 |
| 36.6 | 2.45593 | 17.0 |
| 37.8 | 2.37558 | 10.3 |
| 39.5 | 2.28152 | 13.4 |
| 40.8 | 2.21082 | 10.0 |

In another embodiment, there is provided a crystalline 7-[4-4-naphthalen-1-yl-piperazin-1-yl)-butoxy]-3,4-dihydro-1H-[1,8]naphthyridin-2-one monophosphate having a powder X-ray diffraction pattern shown in FIG. 2 or expressed in terms of 2-theta, d-spacing, and relative intensities all peaks having a relative intensity greater than 10% as provided in Table 2, wherein the powder X-ray diffraction pattern was measured on a Bruker D8 Discover X-ray powder diffractometer with a GADDS CS operating in reflection mode using Cu Kα radiation (1.54). For the sake of identification, this crystalline form of 7-[4-(4-naphthalen-1-yl-piperazin-1-yl)-butoxy]-3,4-dihydro-1H-[1,8]naphthyridin-2-one mono-phosphate is designated as “crystalline Form B or “Form B.

TABLE 2
2-Theta, d-Spacing, and Relative Intensity of All Peaks with Relative
Intensities Greater than 10% for Form B

| 2-Theta | d-Spacing | Relative Intensity (%) |
|---|---|---|
| 10.5 | 8.38518 | 12.6 |
| 11.1 | 7.93180 | 18.5 |
| 12.0 | 7.35124 | 16.1 |
| 13.0 | 13.022 | 13.4 |
| 13.7 | 6.43657 | 46.5 |
| 14.1 | 6.27594 | 33.7 |
| 15.0 | 5.90964 | 38.0 |
| 15.2 | 5.80962 | 47.6 |
| 16.4 | 5.40059 | 19.9 |
| 17.0 | 5.21959 | 59.1 |
| 17.6 | 5.03596 | 63.4 |
| 18.7 | 4.74597 | 23.5 |
| 19.0 | 4.67300 | 25.0 |
| 19.5 | 4.54360 | 33.7 |
| 20.0 | 42958 | 50.8 |
| 20.5 | 4.32225 | 63.3 |
| 21.2 | 4.19346 | 76.4 |
| 22.4 | 3.95711 | 57.1 |
| 23.2 | 3.83075 | 39.9 |
| 23.8 | 3.73944 | 100.0 |
| 24.6 | 3.62189 | 35.6 |
| 25.7 | 3.46002 | 20.0 |
| 26.2 | 3.39437 | 18.7 |
| 27.0 | 3.29722 | 29.4 |
| 27.9 | 3.19756 | 19.5 |
| 28.6 | 3.11999 | 29.4 |
| 29.5 | 3.02741 | 18.1 |
| 30.4 | 2.93860 | 16.7 |
| 31.6 | 2.82900 | 17.0 |
| 32.3 | 2.76828 | 14.8 |
| 33.2 | 2.69857 | 20.6 |
| 33.8 | 2.64718 | 14.4 |
| 35.2 | 2.54717 | 11.9 |
| 36.5 | 2.46231 | 13.0 |
| 37.4 | 2.40289 | 12.4 |

Crystalline Form A and Form B of 7-[4-(4-naphthalen-1-yl-piperazin-1-yl)-butoxy]-3,4-dihydro-1H-[1,8]naphthyridin-2-one mono-phosphate can be identified and distinguished from each other by all the peaks in their respective powder X-ray diffraction patterns as listed in Tables 1 and 2. Form A and Form B may also be identified and distinguished from each other by fewer than all the peaks in their respective powder X-ray diffraction patterns as listed in Tables 1 and 2. For example, Form A may be distinguished from Form B by one or more of the following peaks seen on the powder X-ray diffraction pattern of Form A at 2-theta: 9.7, 14.6, 16.3, 35.8, and 39.5, and maybe further distinguished from Form B by one or more of the following additional peaks seen on the powder X-ray diffiaction pattern of Form A at 2-theta value: 12.4, 12.9, 18.1, 31.1, and 40.8. For a further example, Form A and Form B may be distinguished from each other by one or more of the following peaks seen on the powder X-ray diffraction pattern of Form B at the 2-theta value: 11.1, 13.7, 17.6, 27.0, and 33.2, and maybe further distinguished from each other by one or more of the following additional peaks seen on the powder X-ray diffraction pattern of Form B at the 2-theta value: 14.1, 17.0, 17.6, 19.0, 20.0, and 23.8.

Solid-state stability study demonstrated that crystalline Form A showed little or no degradation at the conditions of 25° C./60% relative humidity, 30° C./70% relative humidity, or 40° C./75% relative humidity, in an open dish over a 6 month time interval. It was further found that crystaine Form A is more stable than Form B.

The X-ray powder diffraction spectra for Form A and Form B, as provided above, were measured on a Bruker D8 Discover X-ray powder diffractometer with GADDS (General Area Diffraction Detector System) CS operating in reflection mode using Cu K_{α} radiation (1.54). The tube voltage and amperage were set to 40 kV and 40 mA, respectively. Scans were collected with the sample to detector distance set at 15.0 cm. The samples were scanned for a period of 60 seconds covering a range of 4.5° to 38.7° in 2θ. The diffractometer was calibrated for peak positions in 2θ using a corundum standard. Samples were run in ASC-6 silicon sample holders. All analyses were conducted at room temperature, which was generally 20° C.-30° C. Data were collected and integrated using GADDS for WNT software version 4.1.14T. Diffractograms were evaluated using DiffracPlus software, release 2003, with Eva version 8.0.

It will be understood that the 2-theta values of the powder X-ray diffraction pattern may vary slightly from one machine to another or from one sample to another, and so the values quoted in Tables 1 and 2 are not to be construed as absolute.

Procedures of performing an X-ray diffraction measurement on a Bruker D8 Discover X-ray powder diffractometer with GADDS CS used for measurements described herein are known in the art. In brief, the sample is typically placed into a cavity in the middle of the silicon sample holder. The sample powder is pressed by a glass slide or equivalent to ensure a random surface and proper sample height. The sample holder is then placed into the Bruker instrument and the powder x-ray diffraction pattern is collected using the instrumental parameters specified above. Measurement differences associated with such X-ray powder diffraction analyses result from a variety of factors including: (a) errors in sample preparation (e.g., sample height), (b) instrument errors, (c) calibration errors, (d) operator errors (including those errors present when determining the peak locations), and (e) the nature of the material (e.g. preferred orientation errors). Calibration errors and sample height errors often result in a shift of all the peaks in the same direction. Small differences in sample height when using a flat holder will lead to large displacements in X-ray powder diffraction peak positions. A systematic study showed that a sample height difference of 1 mm could lead to peak shifts as high as 1020 (Chen et al.; J Pharmaceutical and Biomedical Analysis, 2001; 26, 63). These shifts can be identified from the X-ray diffractogram and can be eliminated by compensating for the shift (applying a systematic correction factor to all peak position values) or recalibrating the instrument. As mentioned above, it is possible to rectify differences in measurements from the various instruments by applying a systematic correction factor to bring the peak positions into agreement. In general, this correction factor will bring the measured peak positions into agreement with the expected peak positions and may be in the range of the expected 2θ value ±0.2°]2θ.

In another aspect of the invention, there is provided a process of for the preparation of crystalline 7-[4-(4-naphthalen-1-yl-piperazin-1-yl)-butoxy]-3,4-dihydro-1H-[1,8]naphthyridin-2-one mono-phosphate salt. The precise conditions under which the crystalline salts are formed may be empirically determined. Processes for preparing crystalline Form A and Form B 7-[4-(4naphthalen-1-yl-piperazin-1-yl)-butoxy]-3,4-dihydro-1H-[1,8]naphthyridin-2-one mono-phosphate salt, which have been found suitable, are described in the Examples hereinafter. For example, crystalline Form A 7-[4-(4-naphthalen-1-yl-piperazin-1-yl)-butoxy]-3,4-dihydro-1H-[1,8]naphthyridin-2-one mono-phosphate may be prepared by the addition of a solution of phosphoric acid in an appropriate solvent, such as water or ethanol, to a solution of 7-[4-(4-naphthalen-1-yl-piperazin-1-yl)-butoxy]-3,4-dihydro-1H-[1,8]naphthyridin-2-one (free base) in an appropriate solvent, such as ethanol or acetonitrile-water, at temperature range from 25° C. to 70° C. The phosphoric acid may be added at once or portion-wise. The mixture may be stirred for an appropriate time and cooled to 0° C. The solid may be collected and dried in a common manner. Crystalline Form B 7-[4-(4-naphthalen-1-yl-piperazin-1-yl)-butoxy]-3,4-dihydro-1H-[1,8]naphthyridin-2-one mono-phosphate may be prepared by addition of a solution of phosphoric acid in an appropriate solvent, such as ethanol, to a solution of 7-[4-(4-naphthalen-1-yl-piperazin-1-yl)-butoxy]-3,4-dihydro-1H-[1,8]naphthyridin-2-one (free base) in an appropriate solvent, such as acetonitrile, at temperature range from 25° C. to 50° C. The phosphoric acid may be added at once or portion-wise. The mixture may be stirred for an appropriate time and cooled to 0° C. or lower. The solid may be collected and dried in a common manner.

Crystalline Form B 7-[4-(4-naphthalen-1-yl-piperazin-1-yl)-butoxy]-3,4-dihydro-1H-[1,8]naphthyridin-2-one mono-phosphate has been found to convert to Form A on standing at ambient temperatures. Therefore, Form A may also be prepared from Form B by allowing Form B to convert to Form A. To prepare Form A from Form B, the Form B materials may be allowed to stand, for example, in an inert atmosphere or in a sealed container, for a period of time, such as two weeks or longer.

In a further aspect of the invention, there is provided a method of treating a disorder or condition in a mammal wherein stimulation of D₂ receptor is beneficial, which method comprises administering to the mammal an effective amount of a crystalline 7-[4-(4-naphthalen-1-yl-piperazin-1-yl)-butoxy]-3,4-dihydro-1H-[1,8]naphthyridin-2-one mono-phosphate salt as described herein above. The invention also relates to the use of a crystalline 7-[4-(4-naphthalen-1-yl-piperazin-1-yl)-butoxy]-3,4-dihydro-1H-[1,8]naphthyridin-2-one monophosphate salt, as described herein above, in the manufacture of a medicament for the treatment of a disorder or condition in a mammal. Pharmacological properties of 7-[4-(4-naphthalen-1-yl-piperazin-1-yl)-butoxy]-3,4-dihydro-1H-[1,8]naphthyridin-2-one mono-phosphate and its use as treatment for CNS disorders, including schizophrenia, can be readily determined using routine methods known in the art. It is preferred that the crystalline 7-[4-(4-naphthalen-1-yl-piperazin-1-yl)-butoxy]-3,4-dihydro-1H-[1,8]naphthyridin-2-one monophosphate salt is crystalline Form A and the mammal is a human. Examples of disorders or conditions that may be treated include major depression, single episode depression, recurrent depression, child abuse induced depression, postpartum depression, dysthymia, cyclothymia and bipolar disorder, schizophrenia, schizoaffective disorder, delusional disorder, substance-induced psychotic disorder, brief psychotic disorder, shared psychotic disorder, psychotic disorder due to a general medical condition, schizophreniform disorder, autism, pervasive development disorder, attention deficit hyperactivity disorder (ADIHD), generalized anxiety disorder, panic disorder, obsessive-compulsive disorder, post-traumatic stress disorder, and phobias, including social phobia, agoraphobia, and specific phobias.

Crystalline 7-[4-(4-naphthalen-1-yl-piperazin-1-yl)-butoxy]-3,4-dihydro-1H-[1,8]naphthyridin-2-one mono-phosphate can be administered to the mammal via any suitable route, such as oral, parenteral (such as subcutaneous, intravenous, intramuscular, intrasternal and infusion techniques), rectal, buccal or intranasal routes. A typical route of administration is oral. It is generally administered in doses ranging from about 3 mg to about 600 mg per day, in single or divided doses (i.e., from 1 to 4 doses per day). The specific amount, however, may vary depending upon the species being treated, weight and condition of the subject being treated and the patient's individual response to said medicament, as well as on the type of pharmaceutical formulation chosen and the time period and interval at which such administration is carried out However, a dosage level that is in the range of about 10 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

Crystalline 7-[4-(4-naphthalen-1-yl-piperazin-1-yl)-butoxy]-3,4-dihydro-1H-[1,8]naphthyridin-2-one monophosphate may be administered alone, or in combination with a pharmaceutically acceptable carrier or diluent. Thus, in another aspect of the invention there is provided a pharmaceutical composition which comprises a crystalline 7-[4-(4-naphthalen-1-yl-piperazin-1-yl)-butoxy]-3,4-dihydro-1H-[1,8]naphthyridin-2-one mono-phosphate salt and a pharmaceutically acceptable carrier. In one embodiment, the pharmaceutical composition comprises crystalline Form A 7-[4-(4-naphthalen-1-yl-piperazin-1-yl)butoxy]-3,4-dihydro-1H-[1,8]naphthyridin-2-one mono-phosphate and a pharmaceutically acceptable carrier. In another embodiment, the pharmaceutical composition comprises crystalline Form B 7-[4-4-naphthalen-1-yl-piperazin-1-yl)-butoxy]-3,4-dihydro-1H-[1,8]naphthyridin-2-one mono-phosphate and a pharmaceutically acceptable carrier. In still another embodiment, the pharmaceutical composition comprises a mixture of crystalline Form A and Form B 7-[4-(4-naphthalen-1-yl-piperazin-1-yl)-butoxy]-3,4-dihydro-1H-[1,8]naphthyridin-2-one mono-phosphate in any ratio and a pharmaceutically acceptable carrier. The pharmaceutical composition may be in the form of a wide variety of different dosage forms, such as tablets, capsules, lozenges, troches, hard candies, suppositories, jellies, gels, pastes, ointments, aqueous suspensions, injectable solutions, elixirs, syrups, and the like. The weight ratio of the novel compounds of this invention to the pharmaceutically acceptable carrier is generally in the range from about 1:6 to about 2:1, and preferably from about 1:4 to about 1:1.

Examples of suitable carriers include solid or liquid diluents, solid fillers, sterile aqueous media, and various non-toxic organic solvents. A variety of carriers may be employed in tablet or other solid dosage forms suitable for oral administration. Examples of suitable carriers for tablets include various excipients such as microcrystalline cellulose, sodium citrate, calcium carbonate, dicalcium phosphate and glycine; disintegrants such as starch (and preferably corn, potato or tapioca starch), alginic acid and certain complex silicates; and binders such as polyvinylpyrrolidone, sucrose, gelatin and acacia. Additionally, lubricating agents such as magnesium stearate, sodium lauryl sulfate and talc are often very useful for tabletting purposes. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

Pharmaceutical compositions for parenteral administration may be in the form of solutions, emulsions, or suspensions. Examples of typical carriers for such compositions include sesame or peanut oil and propylene glycol. The solutions, emulsions, or suspensions should be suitably buffered (preferably pH greater than 8) if necessary and the liquid diluent first rendered isotonic. Generally, these aqueous solutions are suitable for intravenous injection purposes and the oily solutions are suitable for intra-articular, intramuscular and subcutaneous injection purposes. The preparation of all these solutions under sterile conditions is readily accomplished by standard pharmaceutical techniques well known to those skilled in the art.

For intranasal administration or administration by inhalation, crystalline 7-[4-(4-naphthalen-1-yl-piperazin-1-yl)-butoxy]-3,4-dihydro-1H-[1,8]naphthyridin-2-one mono-phosphate may be conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethine, trichlorofluoromethane, dichlorotetrafluoroethane, carbon dioxide or other suitable gas.

### DEFINITIONS

The terms “treatment, “treat, “treating, and the like, refer to alleviating, ameliorating, attenuating, eliminating, reducing, or delaying of the onset of, one or more symptoms of a disorder or condition. These terms also refer to slowing or reversing the progression of a disorder or condition.

The term “therapeutically effective amount” or “effective amount” refers to an amount of a crystalline 7-[4-(4-naphthalen-1-yl-piperazin-1-yl)-butoxy]-3,4-dihydro-1H-[1,8]naphthyridin-2-one mono-phosphate salt being administered sufficient to elicit a pharmacological or therapeutic effect.

The term “mammal” refers to an individual vertebrate animal that is a member of the taxonomic class Mammalia. Examples of mammal includes: humans; companion animals such as cats and dogs; non-human primates such as monkeys and chimpanzees; livestock such as horses, cows, pigs, and sheep; and rodents such as rats, mice, guinea pigs, rabbits, hamsters, and transgenic mice.

The term “pharmaceutically acceptable refers to those substances, compounds, salts, materials, compositions, and/or dosage forms which are, within the scope of sound medical judgment, suitable for use in contact with the tissues of human beings or animals.

The term “carrier as used in connection with a pharmaceutical composition of the invention refers to any substances, compounds, or materials, other than the crystaline 7-[4-(4-naphthalen-1-yl-piperazin-1-yl)-butoxy]-3,4-dihydro-1H-[1,8]naphthyridin-2-one mono-phosphate salt, that are included in a pharmaceutical composition.

### EXAMPLES

The following examples are provided to illustrate certain aspects of the invention, and should not be construed to limit the scope of the claims in any way.

### Example 1

### Preparation of Crystalline Form A 7-r4-(4naphthalen-1-yl-piperazin-1-yl)-butoxy-3,4-dihydro-1H-[1,8]naphthyridin-2-one Mono-phosphate Salt

Crystalline Form A 7-[4-(4-naphthalen-1-yl-piperazin-1-yl)-butoxy]-3,4-dihydro-1H-[1,8]naphthyridin-2-one mono-phosphate salt may be prepared by the following method.

7-[4(4-naphthalen-1-yl-piperazin-1-yl)-butoxy]-3,4dihydro-1H-[1,8]naphthyridin-2-one (181.41 mg) was added to a vial containing 40 mL of acetonitrile. After dissolution of the compound 1.68 mL of water (total of 5% water) was added to the solution. The mixture was mixed well. Then 0.422 mL of 1M phosphoric in water (1:1) was added to the solution. The solution was let slurry at room temperature. Solid precipitated out after 40 minutes.

### Example 2

### Preparation of Crystalline Form A 7-[4-(4-naphthalen-1-yl-piperazin-1-yl)-butoxy]-3,4-dihydro-1H-[1,8]naphthyridin-2-one Mono-phosphate Salt

Crystalline Form A 7-[4-(4-naphthalen-1-yl-piperazin-1-yl)-butoxy]-3,4-dihydro-1H-[1,8]naphthyridin-2-one mono-phosphate salt may also be prepared by the following method.

7-[4-(4-naphthalen-1-yl-piperazin-1-yl)-butoxy]-3,4-dihydro-1H-[1,8]naphthyridin-2-one (5.0 g, 11.62 mmol) was slurried with absolute ethanol (80 mL) in a 250 mL three-neck round-bottom flask equipped with a thermocouple, addition funnel, and mechanical stirrer. The slurry was heated to 70° C. to form a solution and 15.275 M phosphoric acid (0.837 mL, 12.8 mmol, diluted in 12 mL absolute ethanol) was added slowly via the addition funnel in four equal portions. Following the addition of each 14 portion of phosphoric acid, the reaction was stirred at 70° C. for 15 minutes to reform a solution. Upon complete addition of the phosphoric acid, the reaction remained cloudy. This cloudy suspension was stirred at 70° C. for 30 minutes, than cooled to ambient temperature at −5° C./hr. An ice-bath was used to cool the reaction suspension to 0° C. and the solids were filtered cold. Cold ethanol (2×15 mL) was used to wash the filtered crystals, which were dried in the vacuum oven up to 100° C. for several days.

### Example 3

### Preparation of Crystalline Form B of 7-[4-(4-naphthalen-1-yl-piperazin-1-yl)-butoxy-3,4-dihydro-1H-[1,8]naphthyridin-2-one Mono-phosphate Salt

7-[4-(4-naphthalen-1-yl-piperazin-1-yl)-butoxy]-3,4-dihydro-1H-[1,8]naphthyridin-2-one (43 mg, 0.1 mmol) was mixed with acetonitrile (3 mL) and heated to 45° C. in a heating block. A solution of phosphoric acid (1.25 M solution in ethanol, 0.1 mmol −80 μL) at 45° C. was added in two portions. The mixture was stirred at 45° C. for 3 hours, cooled to 30° C. for 1 hour, then cooled to 20° C. and stirred overnight. It was then cooled (in the block) to −8° C. The crystalline solid was filtered out and dried at 50° C./15 Torr for 2.5 hours.

### Example 4

### Effects of 7-[4-(4-naphthalen-1-yl-piperazin-1-yl)-butoxy]-3-dihydro-1H-[1,8]naphthyridin-2-one on Spontaneous Locomotor Activity (SLMA) in Rats

Effects of 7-[4-(4-naphthalen-1-yl-piperazin-1-yl)-butoxy]-3,4-dihydro-1H-[1,8]naphthyridin-2-one on spontaneous locomotors activity (SLMA) was investigated in an in vivo animal model, which is predictive of antipsychotic efficacy.

Method: Male Sprague-Dawley rats were used in studies where SLMA was quantified using an activity monitor in a sound-attenuated chamber. Rats were randomly assigned to various groups (including a vehicle control group). 7-[4-(4naphthalen-1-yl-piperazin-1-yl)-butoxy]-3,4-dihydro-1H-[1,8]naphthyridin-2-one at doses of 0.1 mg/kg, 0.3 mg/kg, 1 mg/kg, 3 mg/kg, and 10 mg/kg, or vehicle were administered orally. Following a 60-minute absorption period, each rat was placed into the test chamber, and SLMA was recorded for a 1-hour time-period. SLMA is expressed as centimeters traveled. Statistical analysis was performed using a one-way ANOVA followed by a post hoc Dunnett's test.

Results: 7-[4-(4-naphthalen-1-yl-piperazin-1-yl)-butoxy]-3,4-dihydro-1H-[1,8]naphthyridin-2-one induced a dose-dependent reduction in SLMA. SLMA, expressed as Mean centimeters traveled in 1 hour±SEM, for the Vehicle group and groups administered with 7-[4-4-naphthalen-1-yl-piperazin-1-yl)-butoxy]-3,4-dihydro-1H-[1,8]naphthyridin-2-one at 0.1 mg/kg, 0.3 mg/kg, 1 mg/kg, 3 mg/kg, and 10 mg/kg was 4066±297, 3998±133, 2510±185, 1977±173, 1742±206, and 1068±208, respectively. SLMA of the compound treated groups, except the 0.1 mg/kg group, was statistically different from the vehicle control group (p<0.05). The minimum effective dose is estimated to be 0.3 mg/kg. These data indicate that 7-[4(4-naphthalen-1-yl-piperazin-1-yl)-butoxy]-3,4-dihydro-1H-[1,8]naphthyridin-2-one is active in inhibiting spontaneous locomotors activity in the in vivo rat model.

1. A crystalline 7-[4-(4-naphthalen-1-yl-piperazin-1-yl)-butoxyl-3,4-dihydro-1H-[1,8]naphthyridin-2-one mono-phosphate.

2. The crystalline 7-[4-(4-naphthalen-1-yl-piperazin-1-yl)-butoxyl-3,4-dihydro-1H-[1,8]naphthyridin-2-one mono-phosphate according to claim 1 having an X-ray powder diffraction pattern with specific peaks at 2-theta: 9.7, 14.6, 16.3, 35.85 and 39.5 measured using Cu Kα radiation.

3. The crystalline 7-[4-(4-naphthalen-1-yl-piperazin-1-yl)-butoxy)-3,4-dihydro-1H-[1,8]naphthyridin-2-one mono-phosphate according to claim 2 having an X-ray powder diffraction pattern with at least one additional peak at 2-theta: 12.4, 12.9. 18.1, 31,1, or 40.8.

4. The crystalline 7-[4-(4-naphthalen-1-yl-piperazin-1-yl)-butoxyl-3,4-dihydro-1H-[1,8]naphthyridin-2-one mono-phosphate according to claim 1 having an X-ray powder diffraction pattern with specific peaks at 2-theta: 9.7, 10.2. 11.6, 12.0, 12.4, 12.9, 14.6, 15.9, 16.3, 18.1, 18.7, 19.3, 19.9, 20.5, 22.1, 23.1, 23.7, 24.4, 25.5, 26.5, 27.5, 28.1, 29.4, 31.1, 31,5 32.9, 34.4, 35.8, 36.6, 37.8, 39.5, and 40.8.

5. The crystalline 7-[4-(4-naphthalen-1-yl-piperazin-1-yl)-butoxyl-3,4-dihydro-1H-[1,8]naphthyridin-2-one mono-phosphate according to claim 1 having an X-ray powder diffraction pattern with specific peaks at 2-theta: 11.1, 13.7, 17.6, 27.0, and 33.2.

6. The crystalline 7-[4-(4-naphthalen-1-yl-piperazin-1-yl)-butoxyl-3,4-dihydro-1H-[1,8]naphthyridin-2-one mono-phosphate according to claim 5 having an X-ray powder diffraction pattern with at least one additional peak at 2-theta: 14.1, 1,70, 17.6, 19.0, 20.0, or 23.8.

7. The crystalline 7-[4-(4-naphthalen-1-yl-piperazin-1-yl)-butoxyl-3,4-dihydro-1H-[1,8]naphthyridin-2-one mono-phosphate according to claim 1 having an X-ray powder diffraction pattern with specific peaks at 2-theta: 10.5, 11.1, 12.0, 13.0, 13.7, 14.1, 15.0, 15.2, 16.4, 17.0, 17.6, 18.7, 19.0, 19.5, 20.0, 20.5, 21.2, 22.4, 23.2, 23.8, 24.6, 25.7, 26.2, 27.0, 27.9, 28.6, 29.5, 30.4, 31.6, 32.3, 33.2, 33.8, 35.2, 36.5 and 37.4.

8. A pharmaceutical composition comprising a crystalline 7-[4-(4-naphthalen-1-yl-piperazin-1-yl)-butoxy]-3,4-dihydro-1H-[1,8]naphthyridin-2-one mono-phosphate salt according to any of claims 1-7 and a pharmaceutically acceptable carrier.

9. A method of treating a central nervous system disorder in a mammal, comprising administering to the mammal a therapeutically effective amount of a crystalline 7-[4-(4-naphthalen-1-yl-piperazin-1-yl)-butoxy]-3,4-dihydro-1H-[1,8]naphthyridin-2-one mono-phosphate salt according to any of claims 1-7.

10. The method according to claim 9, wherein the central nervous system disorder is selected from major depression, single episode depression, recurrent depression, child abuse induced depression, postpartum depression, dysthymia, cyclothymia, bipolar disorder, schizophrenia, schizoaffective disorder, delusional disorder, substance-induced psychotic disorder, brief psychotic disorder, shared psychotic disorder, psychotic disorder due to a general medical condition, schizophreniform disorder, autism, pervasive development disorder, attention deficit hyperactivity disorder, generalized anxety disorder, panic disorder, obsessive-compulsive disorder, post-traumatic stress disorder, and phobias, including social phobia, agoraphobia, and specific phobias.

11. The method according to claim 10, wherein the disorder is schizophrenia or bipolar disorder.

12. The method according to claim 11, wherein the mammal is a human.

13. The method according to claim 12, wherein the crystalline 7-[4-(4-naphthalen-1-yl-piperazin-1-yl-butoxy)-3,4-dihydro-1H-[1,8]naphthyridin-2-one mono-phosphate is the crystalline 7-[4-(4-naphthalen-1-yl-piperazin-1-y)-butoxy]-3,4-dihydro-1H-[1,8]naphthyridin-2-one monophosphate salt according to any of claims 2-4.

14-18. (canceled)

19. The composition according to claim 18, wherein the crystalline 7-[4-(4-naphthalen-1-yl-piperazin-1-yl)-butoxy]-3,4-dihydro-1H-[1,8]naphthyridin-2-one mono-phosphate has an X-ray powder diffraction pattern with specific peaks at 2-theta-9,7, 10,2, 11.6, 12.0, 12.4, 12.9, 14.6, 15.9, 16.3, 18.1, 18.7, 19.3, 19.9, 20.5, 22.1, 23.1, 23.7, 24.4, 25.5, 26.5, 27.5, 28.1, 29.4, 31.1, 31.5, 32.9, 34.4, 35.8, 36.6, 37.8, 39.5, and 40.8.

20. A process for the manufacture of a crystalline 7-[4-(4-naphthalen-1-yl-piperazin-1-yl)-butoxy]-3,4-dihydro-1H-[1,8]naphthyridin-2-one mono-phosphate, comprising addition of phosphoric acid to a solution of 7-[4-(4-naphthalen-1-yl-piperazin-1-yl)-butoxy]-3,4-dihydro-1H-[1,8]naphthyridin-2-one in a solvent.

21. The process according to claim 20, wherein the solvent is ethanol, acetonitrile, or mixture of acetonitrile and water.
19.9, 20.5, 22.1, 23.1, 23.7, 24.4, 25.5, 26.5, 27.5, 28.1, 29.4, 31.1, 31.5, 32.9, 34.4, 35.8, 36.6, 37.8, 39.5, and 40.8.

20. A process for the manufacture of a crystalline 7-[4-(4-naphthalen-1-yl-piperazin-1-yl)-butoxy]-3,4-dihydro-1H-[1,8]naphthyridin-2-one monophosphate, comprising addition of phosphoric acid to a solution of 7-[4-(4-naphthalen-1-yl-piperazin-1-yl)-butoxy]-3,4-dihydro-1H-[1,8]naphthyridin-2-one in a solvent.

21. The process according to claim 20, wherein the solvent is ethanol, acetonitrile, or mixture of acetonitrile and water.
